(19) United States
(12) Patent Application Publication
(10) Pub. No.: US 2024/0285865 A1
(43) Pub. Date: Aug. 29, 2024

(54) SPIRAL ZIPPER-DRIVEN PUMP MECHANISM IN FLUID DELIVERY DEVICE

(71) Applicant: Becton, Dickinson and Company, Franklin Lakes, NJ (US)

(72) Inventors: Dana COTE, Boxford, MA (US); Mark WOOD, Sterling, MA (US)

(73) Assignee: Becton, Dickinson and Company, Franklin Lakes, NJ (US)

(21) Appl. No.: 18/292,532

(22) PCT Filed: Jul. 29, 2022
(86) PCT No.: PCT/US2022/038785

§ 371 Date: Jan. 26, 2024

Related U.S. Application Data
(60) Provisional application No. 63/226,801, filed on Jul. 29, 2021.

(51) Int. Cl. A61M 5/315; A61M 5/142; A61M 5/145

(52) U.S. Cl. CPC A61M 5/31513; A61M 5/14248; A61M 2005/14506

(57) ABSTRACT

A fluid delivery device has a syringe barrel-type reservoir with plunger and plunger driver assembly comprising a spiral zipper-driven pump mechanism. A flexible band in the spiral zipper-driven pump mechanism is transformed from a collapsed, nested configuration to an extended configuration by controllably unfurling and recoiling the band into a spiral column of interlocking revolutions of the band. The column has sufficient stiffness to impart translational motion to a plunger along a longitudinal reservoir axis that is precisely controllable by rotation of gears by a motor. The collapsed, non-extended configuration of the spiral zipper-driven pump mechanism reduces reservoir and fluid delivery device length.

### BACKGROUND

### Field

Illustrative embodiments relate generally to pump mechanisms for use in fluid delivery devices such as wearable medication infusion patches. Illustrative embodiments relate generally to a spiral zipper-driven pump mechanism for controllably extending or retracting a plunger driver in a syringe barrel-type reservoir that does not affect reservoir fluid to ensure biocompatibility, and reduces syringe barrel and drive mechanism length when fully deployed.

### Description of Related Art

Typical drug delivery patch pump designs are challenged by the need achieve small size, low power consumption, accurate delivery, high reliability, and low manufacturing costs. For example, a syringe barrel-type reservoir and associated pump mechanism design can impact overall length of a patch pump and minimizing length of the patch pump form factor is desirable for user wearability. In addition, drug delivery patch pump designs cannot impact drug quality. For example, the materials used for pump mechanism components that contact the delivered fluid cannot present biocompatibility problems.

### SUMMARY

The above and other problems are overcome, and additional advantages are realized, by illustrative embodiments.

Example embodiments of the present disclosure realize several advantages such as minimizing the pump device length, while retaining the beneficial features of highly reliable and proven systems such as pen needles, syringes, or more expensive, non-portable pumping systems that employ a longitudinally extending drive mechanism such as a lead screw-type drive mechanism.

An aspect of illustrative embodiments is to provide an improved and novel spiral zipper-driven pump mechanism design that enables the use of syringe barrel-type drug containers or similar reservoirs, which have been proven to be drug-friendly or biocompatible with drugs and other fluids delivered via fluid delivery devices.

In accordance with illustrative embodiments, a fluid delivery device is provided that has a syringe barrel with a reservoir that can contain fluid and a plunger that translates within the reservoir along a longitudinal reservoir axis thereof to expel fluid from the reservoir and out of the syringe barrel via an outlet. The fluid delivery device comprises a plunger drive assembly mounted at a proximal end of the reservoir. The plunger drive assembly comprises a coiled band coupled to a band drive gear such that, when the band drive gear is rotated in a first rotational direction, the band moves from a nested configuration to an extended configuration that extends from the proximal end of the reservoir toward the distal end of the reservoir and engages the plunger to translate the plunger along the longitudinal reservoir axis toward the distal end of the reservoir. The extended configuration comprises the band uncoiled into a spiral configuration comprising plural revolutions in the band.

In accordance with aspects of the illustrative embodiments, the band is a flexible material chosen from a sheet metal and a plastic film.

In accordance with aspects of the illustrative embodiments, the band comprises slots that are engaged by teeth provided in the band drive gear to controllably extend the band.

In accordance with aspects of the illustrative embodiments, the band is a flat strip of flexible material comprising a top edge and a bottom edge that are each provided with interlocking teeth configured such that the interlocking teeth on a bottom edge of a revolution in the band engage recesses on a top edge of an adjacent revolution in the band.

In accordance with aspects of the illustrative embodiments, the width of the band between its top edge and its bottom edge corresponds to an incremental distance of travel along the longitudinal reservoir axis for each of the revolutions. The band drive gear is configured to be controllably rotated by a motor and drive assembly of the fluid delivery device to extend the band a selected distance determined based on the incremental distance and a selected number of revolutions.

In accordance with aspects of the illustrative embodiments, a distal end of the band is rotatably connected to a back side of a bearing assembly provided at the proximal end of the plunger, and the bearing assembly has a front side that abuts the plunger and applies a force to translate the plunger toward the distal end of the reservoir when the band is extended.

In accordance with aspects of the illustrative embodiments, a distal end of the band is rotatably connected to a back side of a bearing assembly provided at the proximal end of the plunger, and the bearing assembly has a front side connected to the plunger to translate the plunger toward the distal end of the reservoir when the band drive gear is rotated in the first rotational direction to extend the band, and to translate the plunger toward the proximal end of the reservoir when the band drive gear is rotated in a second rotational direction to retract the band.

In accordance with aspects of the illustrative embodiments, the plunger drive assembly further comprises a base receptacle comprising a bottom surface and circumferential side walls for containing the band in its nested configuration. The base receptacle further comprises a slider assembly affixed thereto and extending toward a top opening of the base receptacle. The slider assembly is configured to initially receive a distal end of the band and to surround at least one revolution of the band as the band is extended and before that revolution exits the top opening of the base receptacle while a later revolution is being formed in the slider assembly.

In accordance with aspects of the illustrative embodiments, the band comprises slots that are engaged by teeth provided in the band drive gear to controllably extend the band into the slider assembly and out of the top opening of the base receptacle when the band drive gear is rotated in the first rotational direction.

In accordance with aspects of the illustrative embodiments, the base receptacle further comprises a planetary gear mechanism configured to be rotated when the band drive gear is rotated, the planetary gear mechanism having teeth to engage corresponding teeth provided in the base receptacle to rotate the base receptacle when the band drive gear is rotated.

Additional and/or other aspects and advantages of illustrative embodiments will be set forth in the description that follows, or will be apparent from the description, or may be learned by practice of the illustrative embodiments. The illustrative embodiments may comprise apparatuses and methods for operating same having one or more of the above aspects, and/or one or more of the features and combinations thereof. The illustrative embodiments may comprise one or more of the features and/or combinations of the above aspects as recited, for example, in the attached claims.

### BRIEF DESCRIPTION OF THE DRAWINGS

The above and/or other aspects and advantages of embodiments of the illustrative embodiments will be more readily appreciated from the following detailed description, taken in conjunction with the accompanying drawings, of which:

FIG. 1 is a side view of a wearable fluid delivery device constructed in accordance with an example embodiment;

FIG. 2 is a perspective view of the fluid delivery device of FIG. 1 with the cover removed;

FIG. 3 is a block diagram of example components of a fluid delivery device constructed in accordance with an example embodiment;

FIG. 4 is a partial top view of a spiral zipper-driven pump mechanism constructed in accordance with an example embodiment;

FIGS. 5A and 5B are partial perspective views of a spiral zipper-driven pump mechanism constructed in accordance with an example embodiment;

FIGS. 6A and 6B are partial top views of respective bands configured in accordance with example embodiments; and

FIGS. 7A and 7B are partial side views of a spiral zipper-driven pump mechanism coupled to a plunger in accordance with example embodiments.

Throughout the drawing figures, like reference numbers will be understood to refer to like elements, features and structures.

### DETAILED DESCRIPTION OF ILLUSTRATIVE EMBODIMENTS

As will be appreciated by one skilled in the art, there are numerous ways of carrying out the examples, improvements, and arrangements of a pump in accordance with embodiments disclosed herein. Although reference will be made to the illustrative embodiments depicted in the drawings and the following descriptions, the embodiments disclosed herein are not meant to be exhaustive of the various alternative designs and embodiments that are encompassed by the disclosed technical solutions, and those skilled in the art will readily appreciate that various modifications may be made, and various combinations can be made with departing from the scope of the disclosed technical solutions.

Example embodiments of the present disclosure extend a thin coiled band 80 to a much longer, relatively high strength column 86 to achieve a controlled movement of a syringe plunger 28. This unique design has a very low collapsed height versus extended length. Once coiled into a spiral column 86, the interlocked band has a very high column strength while maintaining precise extension control. Example embodiments realize several advantages such as minimizing the length of fluid delivery device size envelope or form factor, while retaining the beneficial features of highly reliable and proven systems such as pen needles, syringes, or more expensive, non-portable pumping systems that employ a syringe barrel-type plunger drive mechanism such as a lead screw driving mechanism. For example, in accordance with illustrative embodiments described herein, a novel spiral zipper-driven design is employed that enables the use of syringe-based drug containers or similar reservoirs, which have been proven to be drug-friendly or biocompatible with drugs and other fluids delivered via fluid delivery devices. In accordance with example embodiments, a spiral zipper-driven pump mechanism is provided that is controllably extendable and collapsible to reduce length of a syringe barrel-type fluid delivery device.

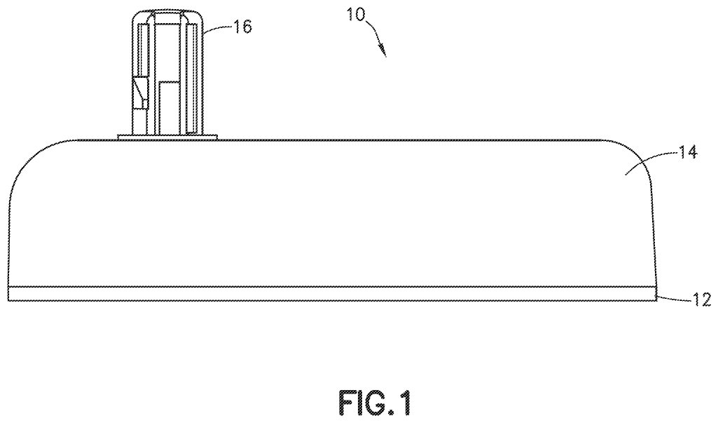

FIG. 1 is a side view of a wearable fluid delivery device 10 constructed in accordance with an example embodiment. The drug delivery device 10 comprises a baseplate 12, a cover 14, and an insertion mechanism 16 in an undeployed position. It is to be understood that the wearable fluid delivery device 10 is being provided as an example syringe barrel-type fluid delivery device for illustrating an example application of spiral zipper-driven pump mechanism constructed in accordance with example embodiments of the present disclosure. The example embodiments of the spiral zipper-driven pump mechanism 30 described herein can also be used with different infusion pumps having different components and configurations, as well as other fluid delivery devices such as autoinjector pens and wearable autoinjectors that use a syringe barrel-style reservoir with plunger.

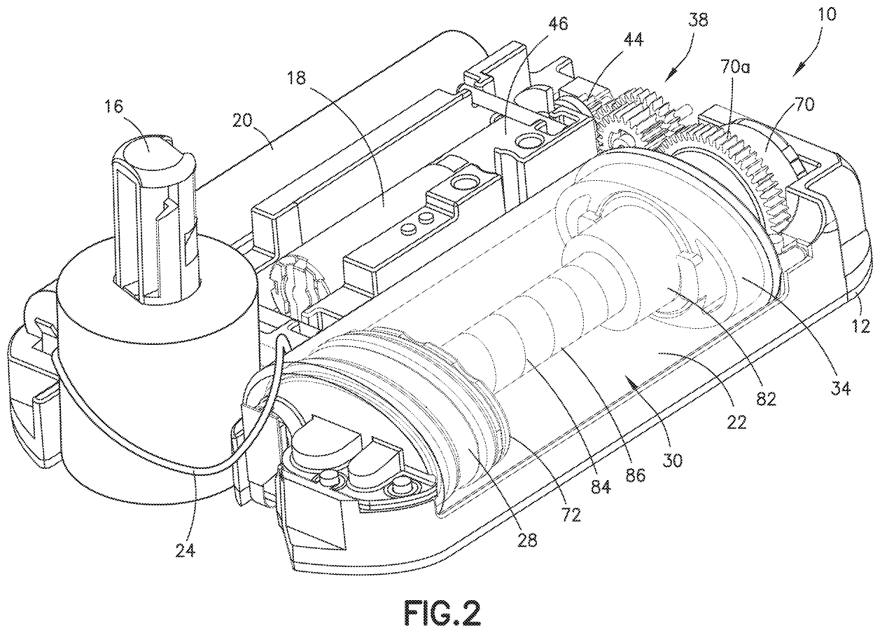

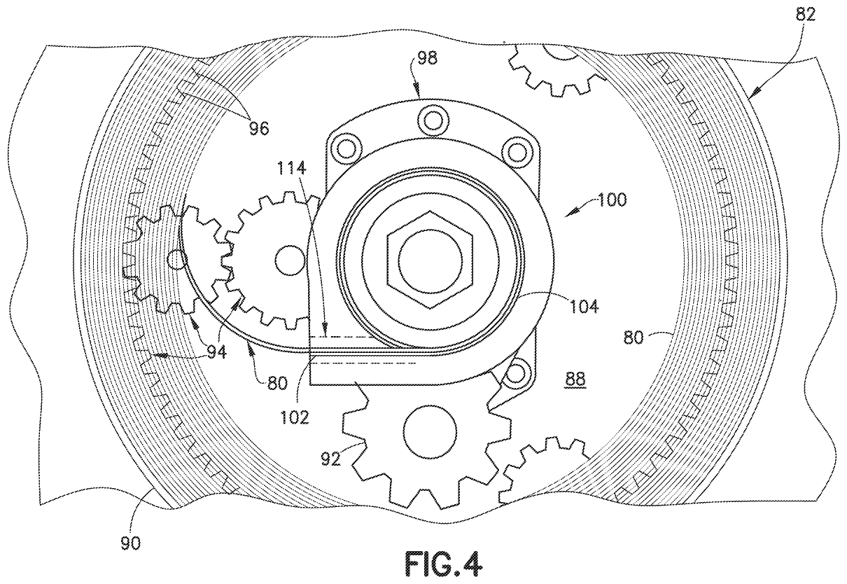

FIG. 2 is a perspective view of the fluid delivery device of FIG. 1 with the cover removed. The baseplate 12 supports the insertion mechanism 16, a motor 18, a power source such as a battery 20, a control board (not shown), and a reservoir 22 or container for storing a fluid to be delivered to a user via an outlet fluid path 24 from and outlet port of reservoir to the insertion mechanism 16. The reservoir 22 can also have an inlet port connected via an inlet fluid path 26 (FIG. 2) to a fill port (e.g., provided in the baseplate 12). The reservoir 22 contains a plunger 28 having a stopper assembly 28. The proximal end of the reservoir 22 is also provided with a plunger driver assembly implemented using a spiral zipper-driven pump mechanism 30 in accordance with example embodiments. The zipper-driven pump mechanism 30 has a band drive gear 92 connected via a gear train 38 to the motor 18. For example, a drive nut 70 can have drive gear teeth 70a that cooperate with teeth on an adjacent gear of the gear train 38 actuated by motor 20 and gearbox 44. The drive nut 70 in turn is configured to cooperate with teeth or other feature of the band drive gear 92, which is provided in a band base receptacle 82 as shown in FIGS. 2 and 4, to impart rotation from the motor 18 to components in the spiral zipper-driven pump mechanism 30. Alternatively, the band drive gear 92 can be a compound gear directly connected to the gear train 38 to impart rotation from the motor 18 to other components in the spiral zipper-driven pump mechanism 30 while obviating the drive nut 70. Although a gear train 38 is shown for illustrative purposes, the drive mechanism can be gears, ratchets, or other methods of inducing rotation from a motor.

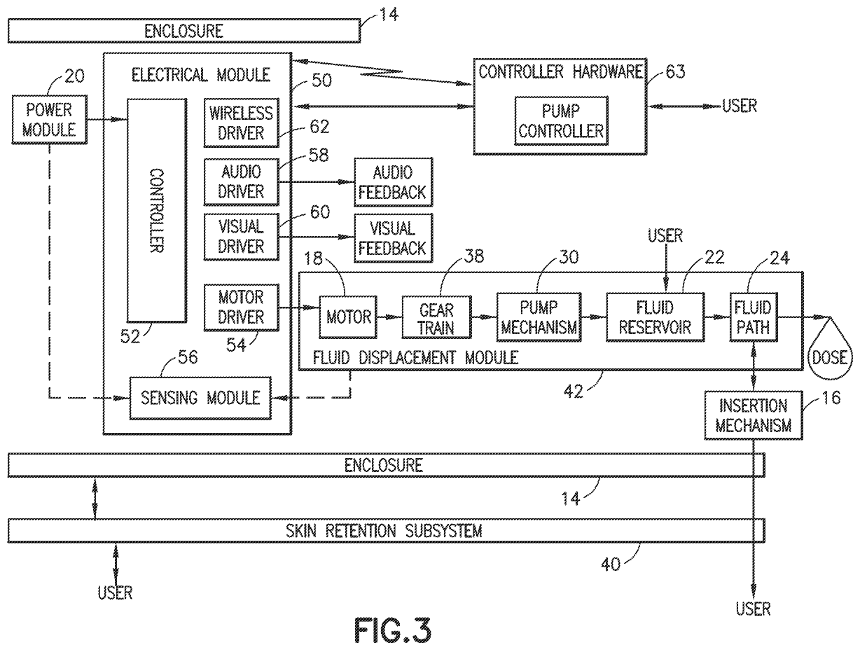

FIG. 3 is a block diagram of example components of a fluid delivery device constructed in accordance with an example embodiment. The cover/housing or device 10 housing is indicated at 14. The device 10 has skin retention subsystem 40 such as an adhesive pad to connect the device 10 to a user's skin. The fluid delivery device 10 further comprises the reservoir 22, the insertion mechanism 16, and a fluid displacement module 42 that can include the motor 18, gear train 38, pump mechanism (e.g., spiral zipper-driven pump mechanism 30), and outlet path 24. The fluid delivery device further comprises electrical components such as a power module (e.g., battery 20), and an electrical module 50 comprising a controller 52, a motor driver 54, optional sensing module 56 to sense fluid flow conditions (e.g. occlusion), optional audio driver 58 (e.g., to indicate dosing in progress, low reservoir, occlusion, successful pairing with external device, or other condition), and an optional visual driver 60, and an optional wireless driver 62 for wireless communication between the fluid delivery device and an optional remote pump control device (e.g., a smartphone or dedicated controller).

With continued reference to FIG. 2, the plunger driver assembly comprising the spiral zipper-driven pump mechanism 30 is shown fully extended with the plunger moved to the distal end of the reservoir 22. It is to be understood that the motor 18 can control the spiral zipper-driven pump mechanism 30 to uncoil and extend incrementally from a fully retracted position to the fully extended position shown to deliver respective designated dose amounts of fluid from a fluid chamber portion 64 of the reservoir 22. As explained below in connection with an illustrative embodiment, the motor 18 and gear train 38 rotate an optional drive nut 70 that in turn can rotate a band drive gear 92 on the spiral zipper-driven pump mechanism 30, or alternatively rotate a gear on the band drive gear 92 if its implemented as a compound gear. The gear train 38 can have different configurations. For example, the gear train 38 can also be in the form of a ratchet indexing mechanism or other indexing mechanism that precisely rotates the drive nut 70 by a mechanically controlled amount. The motor 18 and related gear train components 38 and the optional drive nut 70 can be mounted with respect to each other via a mounting plate or other mechanism secured to the baseplate 12. The reservoir 22 can be secured to the baseplate 12 via a reservoir mount on baseplate (not shown). As shown in FIG. 2, the motor housing 46 secures the motor 18 with respect to the baseplate 12 and the housing and baseplate can be an integral component.

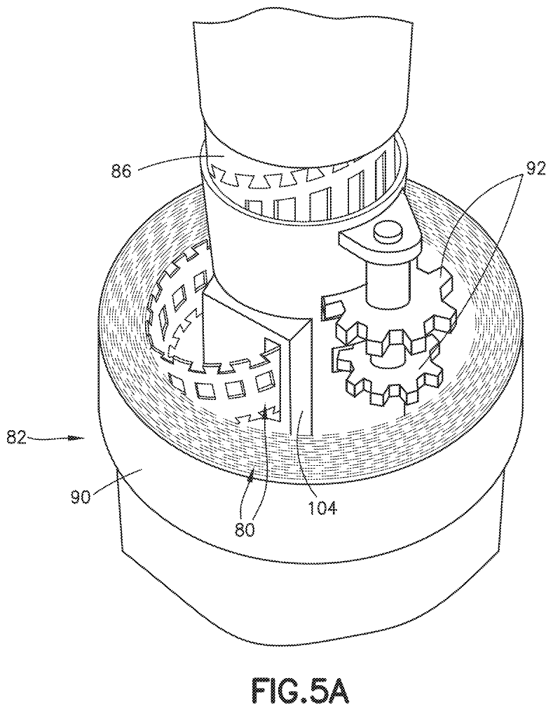

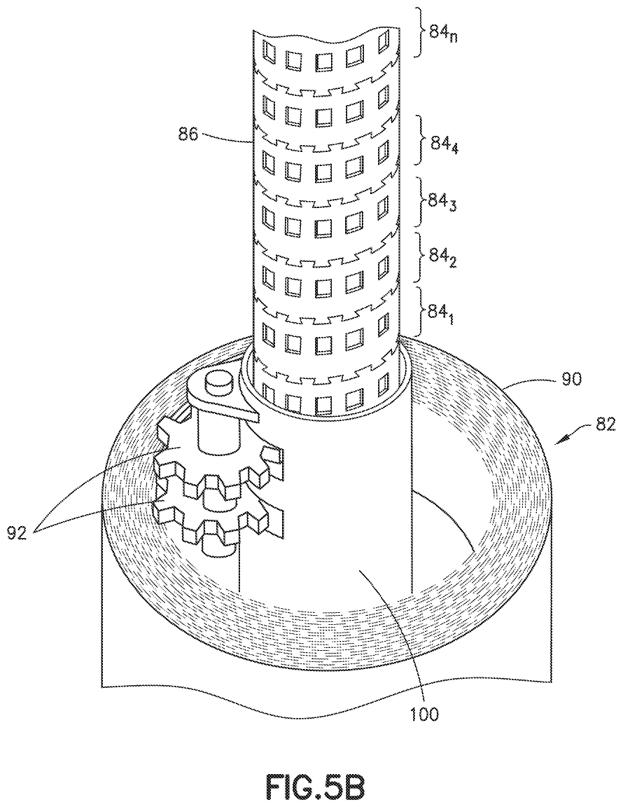

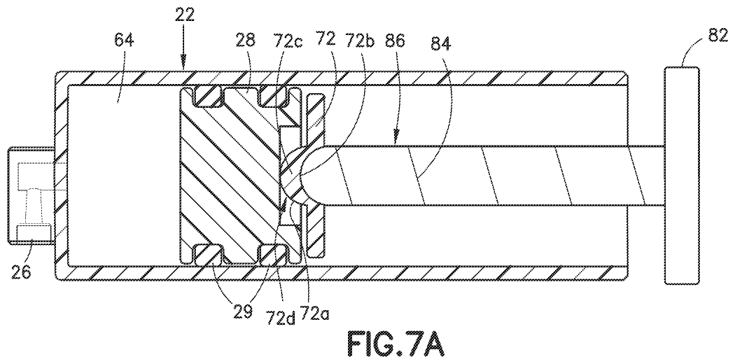

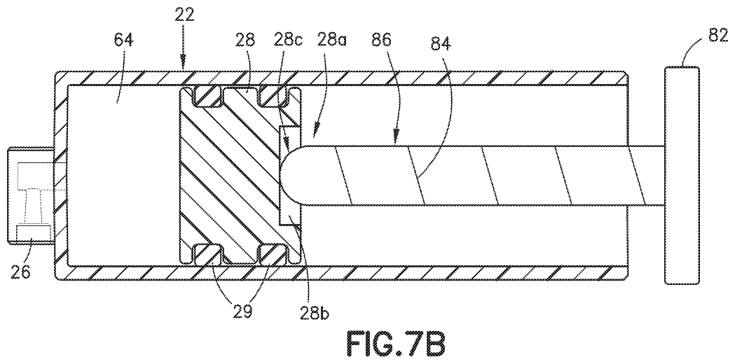

With continued reference to FIGS. 2 and 5A and 5B, an inlet fluid path can be provided from a fill port (not shown) on the underside of baseplate 12 to an inlet port 26 (FIG. 7A) of the reservoir 22 to allow filling of the reservoir prior to shipment, or by a user prior to using the fluid delivery device 10. A gear anchor 34 is provided at a proximal end of the reservoir 22, and is stationary with respect to the reservoir 22. The gear anchor 34 is a disc-shaped member inserted into an opening at the proximal end of the reservoir 22 and can have optional features such as protrusion(s) to facilitate a press or snap fit with respect to the inner wall of the reservoir. The gear anchor 34 has an aperture dimensioned to receive the distal end of the optional drive nut 70. The drive nut 70 is rotationally received in the aperture of the gear anchor 34 and can be connected to impart rotation to the band drive gear 92 of the spiral zipper-driven pump mechanism 30 as the drive nut 70 is rotated to control extension or retraction of a band. As stated above, the band drive gear 92 can alternatively be configured as a compound gear, for example, that is directly connected to the gear train 38 to obviate the drive nut 70 but impart rotation from the motor to other components of the spiral zipper-driven pump mechanism 30. The spiral zipper-driven pump mechanism 30 is provided between the gear anchor 34 and a proximal end of the plunger 28. The plunger 28 is provided within the reservoir 22 and configured to be controllably translated along a longitudinal axis of the reservoir 22 by the joint operations of spiral zipper-driven pump mechanism 30 and the motor 18.

FIGS. 4, 5A and 5B show a spiral zipper-driven pump mechanism 30 in accordance with an example embodiment. With reference to FIG. 4, the spiral zipper-driven pump mechanism 30 comprises a band 80 coiled into a nested configuration within a band base receptacle 82. The band 80 is a length of sheet metal or plastic film or other flexible strip of material that can be retracted and coiled in a nested configuration as shown in FIG. 4, and then extended by unfurling the band 80 and recoiling it into a spiral configuration having a selected number of revolutions and therefore selected length as shown in FIG. 5B. The band material and length are chosen to create an extending column with sufficient stiffness to translate a plunger along a longitudinal axis of a syringe barrel-type reservoir 22 in a fluid delivery device. The length of the band 80 is chosen with at least the design considerations of the diameter and pitch of each revolution, and the overall desired longitudinal axis length to be traveled (e.g., plunger travel distances during lifecycle of reservoir fluid).

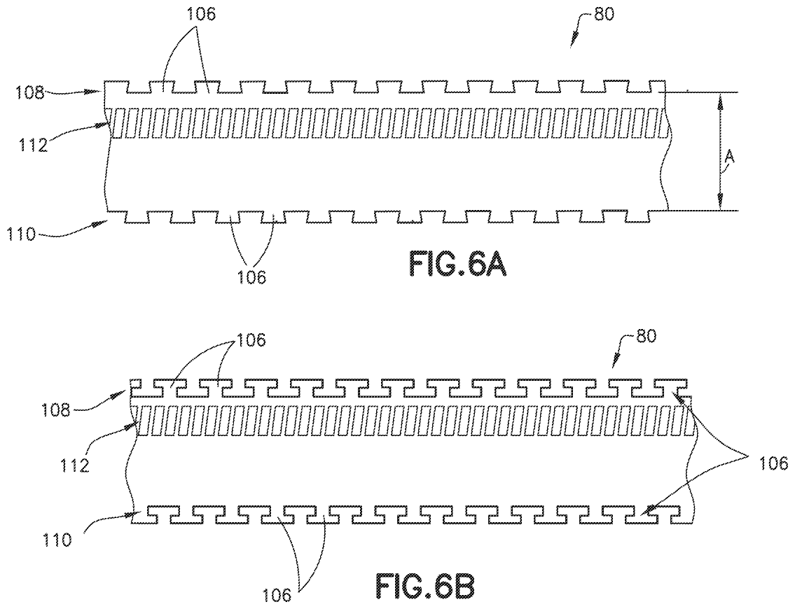

FIGS. 6A and 6B show partial strips of two different example band 80 materials that different in terms of the shape of interlocking teeth 106 provided along the top and bottom edges 108 and 110 thereof. As described below, the band 80 is controllably uncoiled from its nested configuration and recoiled into an extending column 86 of multiple interlocking spiral revolutions (e.g., 84₁, 84₂, . . . , 84ₙ in FIG. 5B), wherein the teeth 106 on a top edge 108 of a revolution (e.g., 84₁) of the band 80 interlock with respective recesses 108 on the bottom edge 110 of an immediately adjacent and previously formed revolution (e.g., 84₂). The band 80 material is selected such that the generated extending column has sufficient stiffness to impart a longitudinal axis translational force on the plunger 28 to translate the plunger 28 a controlled distance to expel a corresponding controlled volume of fluid from the reservoir 22.

As shown in FIGS. 4, 5A and 5B, the band base receptacle 82 of spiral zipper-driven pump mechanism 30 comprises a bottom surface 88 and a side wall 90 to contain the coiled band in the nested configuration and excess band in an extended configuration. The band base receptacle 82 further comprises a fixed base 98 and slider assembly 100 affixed to the bottom surface 88. The slider assembly 100 has side walls of sufficient height to accommodate at least one revolution 84 of the unfurled band 80 after a distal end of the band 80 has been fed into an inlet slot 102. The slider assembly 100 has an internal opening or circumferential groove of a selected diameter corresponding to the desired diameter of the column 86 generated by interlocked revolutions 84 of the band 80 extending from an outlet 104 in the slider assembly 100 toward the plunger 28 in the reservoir 22 under controlled rotations imparted by the motor 18, gear train 38, drive nut 70 and band drive gear 92. As explained below, the distal end of the band 80 can be initially fed into the inlet slot 102 and connected to a connector 28c or 72d for bearing type connected to the plunger 28 or pusher 72, respectively.

With continued reference to FIGS. 4, 5A, 5B, 6A and 6B, teeth in the band drive gear 92 can engage drive slots 112 in the band 80 to advance the band through the slider assembly to form the column 86 as the band drive gear 92 is controllably rotated. To facilitate band 80 extension, the band base receptacle 82 can further comprise a planetary gear 94 and corresponding teeth 96 along the side wall 90. Rotation of the planetary gear 94 can be imparted by rotation of the band drive gear 92. It is to be understood that the placement of the band drive gear 92 and the planetary gear 94 relative to the band base receptacle 82 and slider assembly 100 can be implemented in different configurations than shown, and that different gear mechanism(s) can be used, as understood by one of skill in the art.

The motor 18 and related gear train (e.g., gear train 38, and optional drive nut 70) rotates the band drive gear 92, advancing both the band 80 and the planetary drive system 94. The band 80 advances along a ramp 114 configured within the slider assembly 100 and angled upwardly toward the outlet 104 to set up the desired appropriate angle to move the band up one pitch per revolution 84. After one revolution 84, the band will start to intersect with itself and the band's teeth 106 along edges 108, 110 will start to mesh as described above. The final column 86 height is determined by the number (x) of revolutions multiplied by a width “A” of the band 80 as shown in FIG. 6A. The meshed teeth 106 will maintain the column 86 diameter. The band drive gear 92 simultaneously drives the planetary drive gear 94 and rotates the base receptacle 82 with the unwound band keeping the gears synchronized, and with the rotating advancing column inhibiting binding of the excess band 80.

With reference to FIGS. 7A and 7B, the plunger 28 is decoupled from the band 80 in FIG. 7A, and an intermediate member (e.g., a pusher 72) is used instead to provide an anti-rotation function to minimize off-axis forces on the plunger 28. For example, a bearing interface 72b is provided on the back side of the pusher 72 and can comprise a recess 72c that receives a ball or dome-type connector 72d on the distal end of the band 80 that allows the distal end of the band 80 to rotate within the recess (i.e., from rotation imparted by the band drive gear 92) yet apply translational force along the reservoir longitudinal axis as the band 80 is extended to translate the pusher 72. The pusher 72, in turn, abuts the back side of the plunger 28 to move it toward the distal end of the reservoir 22. In addition, the front side of the pusher 72 can be provided with a ball-joint interface 72a that is received in a recess on the back side of the plunger 28 to minimize off-axis load transfer or forces and any rotation that may be imparted to the plunger 28 from the band 80. An advantage of using the pusher 72 is that its design can be made to reduce off-axis forces that could negatively affect the precision of the motion and overall volume delivery due to uncontrolled plunger 28 wobble. In an alternative embodiment of FIG. 7B, the plunger 28 is not decoupled from the band 80. For example, the distal end of the band 80 can be coupled to the back side of the plunger 28 using a bearing interface 28a (e.g., a recess 28b that receives a ball or dome-type connector 28c on the distal end of the band 80) that allows the distal end of the band 80 to rotate via the band drive gear 92 yet apply translational force along the reservoir longitudinal axis as the band 80 is extended.

The plunger 28 can have a stopper assembly to prevent leakage of any fluid retained in a fluid chamber portion of the reservoir 22. For example, the plunger 28 can be configured to have one or more (e.g., two) circumferential groove dimensioned to accommodate respective O-ring(s) 29. For example, using two O-rings 29 increases stability (e.g., even in spite of an increase in length). Depending on dose accuracy requirements, a single O-ring can be a viable option; however, for high precision, two O-rings are particularly beneficial. Alternatively, the stopper assembly can comprise, for example, an elastic member comprising elastic material similar to a syringe stopper and configured as disc mounted to a surface of a plunger disc or as a band of material surrounding the plunger disc.

The spiral zipper-driven pump mechanism 30 provides a technical solution to technical problems of accurately moving a plunger and dispensing fluid from a chamber and reducing form factor of fluid delivery devices. The spiral zipper-driven pump mechanism 30 is similar to that of a screw driving design. For example, it will advance one pitch per revolution. The spiral zipper-driven pump mechanism 30 however takes up much less space in the longitudinal reservoir axis in its collapsed form. More specifically, the configuration of the spiral zipper-driven pump mechanism 30 components with respect to the reservoir 22 and the plunger 28 realizes a number of advantages. For example, having spiral zipper-driven pump mechanism 30 mounted at a proximal end of the reservoir 22 and having a collapsed configuration that minimizes extension into the reservoir until the band drive gear 92 is rotated optimizes use of the reservoir chamber for fluid delivery instead of having to accommodate longer pre-delivery plunger driver components. In addition, the overall length of the reservoir can be substantially the same as the length of the housing, with the addition of a small amount of headspace to accommodate the band base receptacle 82 and its connection to the gear train 38. Thus, the overall longitudinal footprint of the pump mechanism and the longitudinal axis dimension of the fluid delivery device housing can be reduced. The use of the plunger 28 and plunger drive assembly comprising the spiral zipper-drive mechanism 30 also minimizes contact of the pump mechanism with the fluid being delivered to ensure biocompatibility between the fluid and the fluid delivery housing.

The example embodiments described herein employ an elliptical syringe barrel-type reservoir 22 to contain the drug or fluid to be delivered. The elliptical syringe barrel-type reservoir 22 provides anti-rotation functionality and associated benefits. For example, anti-rotation provided by the intrinsic design of an elliptical syringe barrel-type reservoir 22 naturally prevents rotation of the barrel when a torque is applied. The elliptical shape also has the added benefit of potentially saving overall device height.

Reservoir 22 can be configured to be durable, that is, not removable but rather preinstalled within the fluid delivery device housing 14. The reservoir 22 can be similar in materials to a syringe barrel and associated stopper. The reservoir 33 can be prefilled and the spiral zipper-driven mechanism 30 and plunger 28 initially in a retracted position. Alternatively, the fluid delivery device housing 14 can be provided with a fill port and fluid path 26 from the fill port to the reservoir 22. The fill port can be configured for filling by a user with a syringe, or by using a filling station that fluidically couples to the fill port. The spiral zipper-driven mechanism 30 is initially in its retracted position but the plunger 28 can be in an initial deployed position (e.g., located at the distal end of the reservoir 22 or a position between the distal end and the fully retracted spiral zipper-driven mechanism 30 such that sufficient filling volume is needed in the reservoir 22 to push the plunger 28 toward abutment with the front face of the pusher 72 if used in lieu of a direct connection between a bearing interface 28b of the plunger 28 and spiral zipper-driven mechanism 30.

Although various persons, including, but not limited to, a patient or a healthcare professional, can operate or use illustrative embodiments of the present disclosure, for brevity an operator or user is referred to as a “user” herein.

Although various fluids can be employed in illustrative embodiments of the present disclosure, for brevity the liquid in a fluid delivery device is referred to as “fluid” herein.

It will be understood by one skilled in the art that this disclosure is not limited in its application to the details of construction and the arrangement of components set forth in the above description or illustrated in the drawings. The embodiments herein are capable of other embodiments, and capable of being practiced or carried out in various ways. Also, it will be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted,” and variations thereof herein are used broadly and encompass direct and indirect connections, couplings, and mountings. In addition, the terms “connected” and “coupled” and variations thereof are not restricted to physical or mechanical connections or couplings. Further, terms such as up, down, bottom, and top are relative, and are employed to aid illustration, but are not limiting.

The components of the illustrative devices, systems and methods employed in accordance with the illustrated embodiments can be implemented, at least in part, in digital electronic circuitry, analog electronic circuitry, or in computer hardware, firmware, software, or in combinations of them. These components can be implemented, for example, as a computer program product such as a computer program, program code or computer instructions tangibly embodied in an information carrier, or in a machine-readable storage device, for execution by, or to control the operation of, data processing apparatus such as a programmable processor, a computer, or multiple computers.

The above-presented description and figures are intended by way of example only and are not intended to limit the illustrative embodiments in any way except as set forth in the following claims. It is particularly noted that persons skilled in the art can readily combine the various technical aspects of the various elements of the various illustrative embodiments that have been described above in numerous other ways, all of which are considered to be within the scope of the claims.

1. A fluid delivery device with a syringe barrel having a reservoir that can contain fluid and a plunger that translates within the reservoir along a longitudinal reservoir axis thereof to expel fluid from the reservoir and out of the syringe barrel via an outlet, the fluid delivery device further comprising:
a plunger drive assembly mounted at a proximal end of the reservoir and comprising a coiled band coupled to a band drive gear such that, when the band drive gear is rotated in a first rotational direction, the band moves from a nested configuration to an extended configuration that extends from the proximal end of the reservoir toward the distal end of the reservoir and engages the plunger to translate the plunger along the longitudinal reservoir axis toward the distal end of the reservoir;
wherein the extended configuration comprises the band uncoiled into a spiral configuration comprising plural revolutions in the band.

2. A fluid delivery device as claimed in claim 1, wherein the band is a flexible material chosen from a sheet metal and a plastic film

3. A fluid delivery device as claimed in claim 1, wherein the band comprises slots that are engaged by teeth provided in the band drive gear to controllably extend the band.

4. A fluid delivery device as claimed in claim 1, wherein the band is a flat strip of flexible material comprising a top edge and a bottom edge that are each provided with interlocking teeth configured such that the interlocking teeth on a bottom edge of a revolution in the band engage recesses on a top edge of an adjacent revolution in the band.

5. A fluid delivery device as claimed in claim 4, wherein the width of the band between its top edge and its bottom edge corresponds to an incremental distance of travel along the longitudinal reservoir axis for each of the revolutions, the band drive gear configured to be controllably rotated by a motor and drive assembly of the fluid delivery device to extend the band a selected distance determined based on the incremental distance and a selected number of revolutions.

6. A fluid delivery device as claimed in claim 1, wherein a distal end of the band is rotatably connected to a back side of a bearing assembly provided at the proximal end of the plunger, the bearing assembly having a front side that abuts the plunger and applies a force to translate the plunger toward the distal end of the reservoir when the band is extended.

7. A fluid delivery device as claimed in claim 1, wherein a distal end of the band is rotatably connected to a back side of a bearing assembly provided at the proximal end of the plunger, the bearing assembly having a front side connected to the plunger to translate the plunger toward the distal end of the reservoir when the band drive gear is rotated in the first rotational direction to extend the band, and to translate the plunger toward the proximal end of the reservoir when the band drive gear is rotated in a second rotational direction to retract the band.

8. A fluid delivery device as claimed in claim 1, wherein the plunger drive assembly further comprises a base receptacle comprising a bottom surface and circumferential side walls for containing the band in its nested configuration, the base receptacle further comprising a slider assembly affixed thereto and extending toward a top opening of the base receptacle, the slider assembly configured to initially receive a distal end of the band and to surround at least one revolution of the band as the band is extended and before that revolution exits the top opening of the base receptacle while a later revolution is being formed in the slider assembly.

9. A fluid delivery device as claimed in claim 1, wherein the band comprises slots that are engaged by teeth provided in the band drive gear to controllably extend the band into the slider assembly and out of the top opening of the base receptacle when the band drive gear is rotated in the first rotational direction.

10. A fluid delivery device as claimed in claim 9, wherein the base receptacle further comprises a planetary gear mechanism configured to be rotated when the band drive gear is rotated, the planetary gear mechanism having teeth to engage corresponding teeth provided in the base receptacle to rotate the base receptacle when the band drive gear is rotated.
